(19) United States
(12) Patent Application Publication
(10) Pub. No.: US 2017/0258507 A1
(43) Pub. Date: Sep. 14, 2017

(54) SINGLE HAND INJECTION CONTROL DEVICE WITH A SELF-ADJUSTING POSITION REFERENCE

(71) Applicant: Hugh Hetherington, Bozeman, MT (US)

(72) Inventor: Hugh Hetherington, Bozeman, MT (US)

(21) Appl. No.: 15/608,861

(22) Filed: May 30, 2017

Related U.S. Application Data
(63) Continuation-in-part of application No. 14/025,933, filed on Sep. 13, 2013.
Application No. 14/025,933 is a continuation-in-part of application No. 13/734,974, filed on Jan. 5, 2013, now abandoned.
Application No. 13/734,974 is a continuation of application No. 12/285,203, filed on Sep. 30, 2008, now abandoned.
Application No. 12/285,203 is a continuation-in-part of application No. 12/078,603, filed on Apr. 2, 2008, now Pat. No. 8,133,208.

(51) Int. Cl. A61B 17/88; A61B 5/00; A61M 5/32; A61M 5/20; A61M 5/315

(52) U.S. Cl. CPC A61B 17/8822; A61M 5/20; A61M 5/31511; A61M 5/31576; A61M 5/3158; A61M 5/31585; A61M 5/3287; A61B 5/0002; A61B 2017/00747

(57) ABSTRACT

A single hand operable injection control device (ICD) automatically injects material into a subject tissue at a rate controllably proportional to the rate of movement of the syringe. Squeezing the figure holds rotates a transmission/timing belt that is connected to a position reference member/guide that pushes against the subject tissue, causing the ICD body (containing the syringe) to push away from the subject tissue. The timing belt turns a worm gear that drives a syringe plunger into the syringe held by the ICD. The coordinated motions result in precise delivery of a unit volume/region of injectate into the subject. That is, a uniform “tube” of injectate is automatically laid in the cannula track.

### CROSS REFERENCE TO RELATED APPLICATIONS

This application is a Continuation-In-Part of pending U.S. patent application Ser. No. 14/025,933 filed Sep. 13, 2013, which is a Continuation-In-Part of U.S. patent application Ser. No. 13/734,974 filed Jan. 5, 2013 (and now abandoned), which is a Continuation-In-Part of U.S. patent Ser. No. 12/285,203 filed Sep. 30, 2008 (and now abandoned) which is a Continuation-In-Part of U.S. patent application Ser. No. 12/078,603, filed Apr. 2, 2008, now issued as U.S. Pat. No. 8,133,208 on Mar. 13, 2012, and claims benefit to the priorities thereof. The contents being incorporated herein by reference in their entirety.

### FIELD

This disclosure relates to an injection or extraction device, referred to hereafter as the injection control device (ICD). More particularly, this disclosure relates a single hand useable design that allows the automatic injection (into subject) or extraction (from subject), a proportional volume of material from the syringe as a function of cannula displacement.

### BACKGROUND

In the cosmetic surgery arts, as one example, there is the requirement for placement (extraction) of material, for example, a filler material or fat cells, etc. in a patient in a manner that leaves (withdraws) a defined thickness or consistent profile of material per unit region (or pass). That is, the channel created by the cannula's insertion (extraction) is filled (removed) with the filler material. The practitioner must move the cannula at a precise rate (aka—a pass) while coordinating the rate of injecting (extracting) the material to match the cannula's rate of movement, hopefully avoiding causing lumping (too much) or sinking (too little) in treated region. Too little or too much displacement of material (or inconsistent cannula movement) causes an unnatural appearance in the skin or other treated areas of the body.

In particular, the traditional method is to manually withdraw or inject the cannula of the syringe into the subject while “manually” pressing (with the thumb) the syringe's plunger in synchronicity. Of course, it goes without saying this approach is sensitive to the practitioner's skill level and produces different results for different passes. Being unavoidably subject to human error, inconsistent results (e.g., lumps, thin lines, voids, etc.) often occur, as well as possible damage to the patient. (Imagine the scenario where multiple adjoining passes are made in a patient's face, and then one or more neighboring track(s) are clearly thicker (or lumpy) than the others—a very undesirable result.) Accordingly, there has been a long-standing need in the discipline to devise systems and methods for addressing the problems discussed above.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In accordance with one aspect of the present disclosure, an injection control device is provided, comprising: a body have a forward and rearward end; a syringe supporting section coupled to the forward end of the body; a gear assembly coupled to the body, and configured to actuate a prospective syringe plunger when the gear assembly is driven; a transmission system coupled to the body and to the gear assembly, providing a driving force when actuated; at least one transmission system actuator, coupled to the transmission system; an extendable, automatically self-positioning, position reference member (PRM), a forward portion having a reference end for contact with a subject surface and extending from the forward end of the body; and a transmission-to-PRM engagement system coupled to the transmission system and coupled to the PRM, wherein when the actuator is motioned in a designated direction, the transmission system drives the gear assembly to actuate the prospective syringe plunger into a prospective syringe barrel held in the syringe supporting section and drives the transmission-to-PRM engagement system to actuate the position reference member against the subject surface, causing the body and prospective syringe to move away from the subject surface, these combination of motions resulting in an injectate to be deposited in proportion to a function of the position reference member travel or prospective syringe cannula travel.

In accordance with another aspect of the present disclosure, the injection control device of above is provided, wherein the transmission is comprised of a pulley coupled to the rearward end of the body, and at least one timing belt coupled to the gear assembly and to the pulley; and/or wherein the transmission-to-PRM engagement system is comprised of: a pawl with an angled end directed to the forward end of the body, coupled to the transmission system, configured to move forward when the transmission system is activated via motion of the actuator; and a ramp coupled to the body, causing the pawl, when moved forward, to engage the PRM; and/or wherein a PRM has angled teeth; and/or further comprising a syringe with a cannula held by the injection control device; and/or further comprising: at least one spring coupled to the transmission system and the PRM; and a gear assembly release, wherein as the actuator is released, the actuator and the PRM automatically return to a starting position and the prospective syringe plunger remains stationary; and/or further comprising a toothed syringe plunger in contact with the gear assembly; and/or wherein the gear assembly comprises a worm and worm gear; and/or wherein the worm is a fixed direction worm; and/or wherein the fixed direction is via a clutch; and/or further comprising a spring in the pulley, providing a restorative force to bring the actuator into an initial resting position; and/or wherein the actuator is adapted to fit a finger and configured to travel though a guide slot in the body; and/or wherein the timing belt is a plurality of timing belts; and/or further comprising a thumb hold disposed at the rearward end of the body; and/or wherein the reference end of the PRM is positioned over the prospective cannula; and/or wherein actuator enables operation with a single hand; and/or wherein at least one of the pawl and ramp functions as a ratchet; and/or wherein the injectate is at least one of a fat, filler, flavor, oil, liquid, living cells, and chemical; and/or further comprising: a guide slot in the PRM; and a stop disposed in the body and in the guide slot, controlling a range of motion of the PRM; and/or wherein the stop is removable.

### BRIEF DESCRIPTION OF THE DRAWINGS

FIG. 1 is an illustration of a perspective view of an exemplary injection control device (ICD) with a syringe.

FIG. 2 is an illustration of the side view of the exemplary ICD, with the upper portion of the timing belt that is coupled to the finger actuators visible through the slot.

FIG. 3 is a cut-away illustration of the illustration of FIG. 2, showing the internal components of the ICD.

FIG. 4 is a top perspective view of the front and rear chassis of the ICD with the principal mechanics exposed.

FIG. 5 is a close-up side view of the pawl to ramp arrangement.

FIG. 6 is an exposed partial view of the timing belts with finger actuators and thumb rest.

FIG. 7 is a reversal of FIG. 4, with a close-up view of the worm gear to timing belt to main gear configuration.

### DETAILED DESCRIPTION OF THE DRAWINGS

The claimed subject matter is now described with reference to the drawings. Reference to the above incorporated application(s)/patent(s) provide extensive descriptions on the mechanics for achieving semi-automatic, rate-specific delivery/extraction. In particular, the incorporated embodiments are shown where, in most instances, the positioning guide is manually extended/operated to establish a reference position for when the cannula and plunger are simultaneously operated in synchronicity (i.e. coordinated via mechanical means to move in direct proportion to each other). For example in an injection scenario, the positioning guide is manually “held” in position “against” the skin, by the practitioner while the injection control device (ICD) is being pulled away (toward) during operation. This typically requires two hands to operate.

In various embodiments as now described in this disclosure, the configuration of the injection control device (ICD) is such that the need to manually extend/operate the positioning guide and hold it during the ICD operation is obviated, allowing for single hand operation. Quite briefly, the distal end of the positioning guide is automatically positioned into proximity with the cannula tip and “retracts” as it touches the subject tissue, while the cannula is first inserted “into” the tissue (injection scenario). Thereafter, when the practitioner withdraws the cannula “from” the tissue (to initiate the injection of the material), the motion of withdrawing triggers a transmission that translates the motion to pushing against syringe's plunger, thus coordinating the rate of material injection with the rate of cannula withdrawal. Specific aspects of the various modifications and improvements are detailed in the following FIGS.'s descriptions.

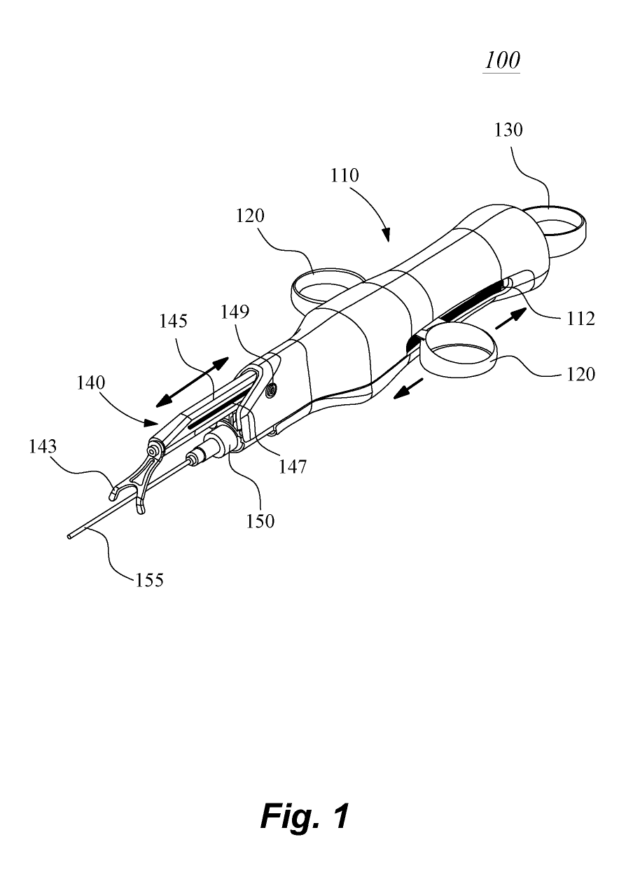

FIG. 1 is an illustration of a perspective view 100 of an exemplary injection control device (ICD) with a syringe. The ICD has a body 110 with openings 112 that allow for actuators 120 (shown here to accommodate placement of one or more fingers) so travel therein (indicated here with arrows). Body 110 is terminated at its rear with a thumb rest 30 and at its front end with a “springed” position reference member 140 that automatically travels in and out of the body's 110 cannula-side opening. Position reference member 140 is composed of a travel arm 145 with a travel guide 147, which is shown here as a slot within the travel arm 145; and reference tip 143. Pin 149 in body 110 extends into the travel guide/slot 147 and acts as a stop to prevent the travel arm 145 from extending completely out of the body 110 (from forces from the internal spring—not shown). Reference tip 143 provides a tissue contact area and while shown here as having two fingers, more or less fingers may be used as well as different shapes. For example, a bent plate with a hole for the cannula may be used or a curved circular ring. Therefore, it should be understood other styles, designs for the reference tip 143 may be used without departing from the spirit and scope of this disclosure. In some embodiments, the entirety of the position reference member 140 may be a single piece. Syringe 150 is shown positioned inside the ICD body 110 and cannula 155 optionally extends from the ICD body 110 past the reference tip 143. In some embodiments, the syringe 150 may be situated at different positions within the ICD body 110.

In an injection operation, the exemplary ICD's position reference member 140 is automatically extended (and may be further extended than shown in FIG. 1, or less extended) via an internal springing mechanism, into its preliminary position. The operator holds the ICD body 110 (for example, via finger actuators 120 and thumb rest 130, or otherwise) and inserts the cannula tip 155 into the subject. As the cannula tip 155 is advanced into the subject, the reference tip 143 (and position reference member 140), encounters the tissue surface, and will be pushed back towards the ICD body 110 until the reference tip 143 arrives at its final position (for example, when the operator stops advancing the cannula).

Thereupon, the operator is ready to inject a “track” of material, wherein the ICD's drive/transmission mechanism (not shown) is triggered by manually squeezing the finger actuators 120 towards the thumb rest 130. This action causes the internal plunger (not shown) to be advanced into the barrel of the syringe 150 via an internal worm gear (not shown), and importantly the position reference member 140 to lock to the drive/transmission and actively extend from the body 110 of the ICD causing the body 110 to be pushed away from the subject which, in turn simultaneously causes the cannula to be withdrawn from the subject. Consequently, the plunger will be advanced in a consistently proportional ratio depositing a thread of injectate that is of uniform volume per unit distance of cannula travel and/or position reference member 140 travel. This exchange of action is initiated via an internal transmission system (shown in this disclosure, for example, as a timing belt)—which is connected to the finger actuators 120, motions the syringe plunger and drives it into the syringe 150 thus, expressing material from the syringe 150. It should be understood that the displacement of the syringe 150 is in direct proportion to the displacement of the position reference member 140, so their opposite motions are related to each other.

When pressure on the finger actuators 120 is released, a return system (for example, a spring) causes the finger actuators 120 to return to the starting position which, in turn causes the position reference locking mechanism to be released, allowing the position reference member 140 to be passively extended to the forward resting position. A clutching mechanism in the drive train allows the finger actuators 120 to return to the passive position without causing the plunger to move relative to the syringe 150. While FIG. 1 describes the finger actuators 120 as the “moving” part, an alternative embodiment could have the finger actuators 120 fixed to body of the device and the thumb rest 130 or butt plate to move relative to the body 110 of the ICD.

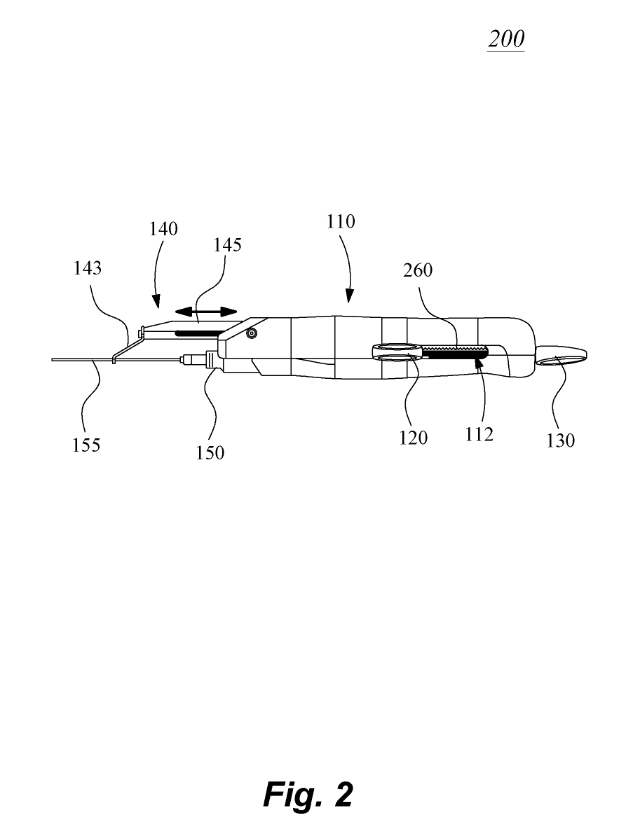

FIG. 2 is an illustration of the side view 200 of the exemplary ICD. Here, the upper portion of the timing belt 260 that is coupled to the finger actuators 120 is visible through the openings 112. In the embodiments described here, the timing belt 260 is actually a pair of timing belts, forming two separate but parallel “loops,” however more or less loops, or different sized loops, configurations may be used, according to design preference.

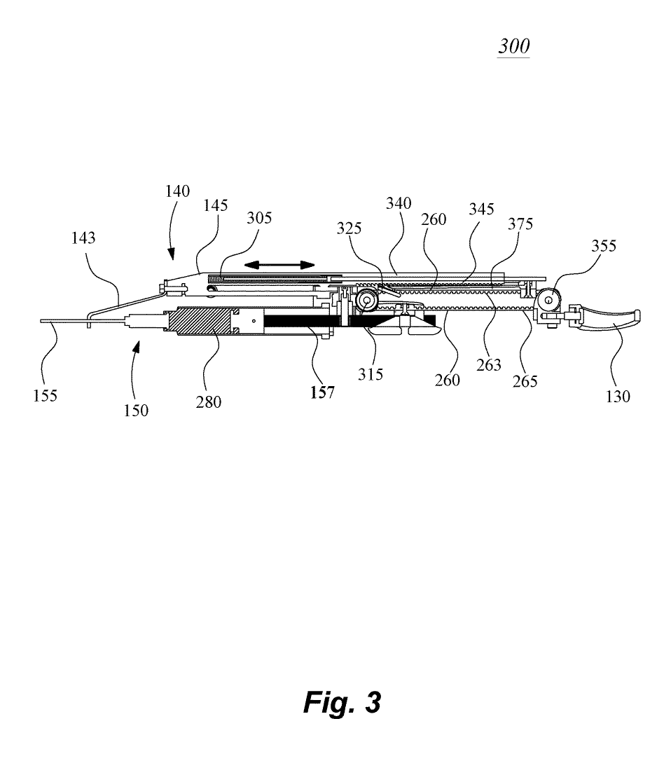

FIG. 3 is a cut-away illustration 300 of the illustration of FIG. 2, showing the internal components of the ICD. Beginning with the simplest object, the syringe 150 contains injectate 280 within its barrel, terminated with the cannula 155 and the plunger 157. Plunger 157 is coupled to worm 315 via a toothed edge (not shown) of the plunger 157. Therefore, when the worm 315 is engaged, its interaction with the plunger's toothed edge causes the plunger 157 to move. The worm 315 is motioned by the teeth of 263 of timing belt 260, which rotates, on one end, about timing pulleys 355 affixed to the ends of the axle of worm 315 and, on the other end, about a pulley 355. The actual rotation of the timing belt 260 is caused by the force exerted by the operator on the finger actuators 120, which are fixed to a section of the timing belt 260 (via a pin, or screw, or clasp, etc.). Thus, squeezing together the finger actuators 120 and thumb rest 130 causes the finger actuators 120 to slide rearward, pulling the timing belt 260 with it.

As will be detailed later, the squeezing motion (or some facsimile) also engages a linear clutching mechanism composed, in an embodiment, of a pawl 375 connected to the timing belt 260, that slides the pawl's 375 end into a ramp 325 to direct the pawl 375 to one of the angled teeth 345 of a rear portion 340 of the position reference member 140. The squeezing motion thus “pushes” the position reference member 140 out from the mouth of the ICD. Specifically, the timing belt 260 and pawl 375 arrangement translates the pressure from the “squeezing” motion to “push” against the position reference member 140 to cause the ICD body to pull away (e.g., retract) from the tissue surface—exposing more and more of the position reference member 140 from the ICD as the ICD body is retracted. Since the syringe 150 is connected to the ICD body, it too will travel with the ICD body, bringing the cannula 155 with it. Conversely, as the cannula 155 is traveling “rearward” with the ICD body, the plunger 157 travels “forward” to inject the injectate via the worm 315/timing belt 260 interaction. The combined action of the plunger 157 being pushed into the barrel of the syringe 155 and the body of ICD being pushed away from the subject causes injectate to be deposited in the track of the cannula 155 in a volume that is consistently proportional to the distance traveled by the cannula 155, or alternatively, the position reference member 140.

Spring 305 is used to push the position reference member 140 back to its initial position after use. A clock spring or some similarly functioning mechanism in either the worm 315 or pulley 355 can provide forces to the finger actuators 120 to bring them back to their resting position, after operation. This can all occur while having the plunger 157 “disengaged” via a clutch or unidirectional bearing, for example, within the transmission system, so that the plunger 157 is stationary within the syringe 155 barrel. The range of travel of the position reference member 140 can be adjusted by the use of removal “stops” (for example, 149 in FIG. 1). The position reference member 140, with its angled teeth 345, can be designed to act as a ratchet, being restricted from return travel when being engaged.

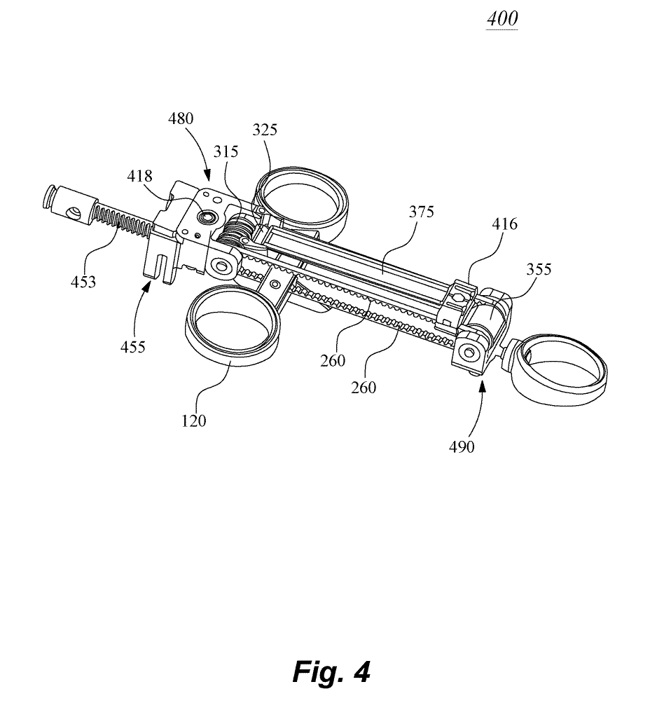

FIG. 4 is a top perspective view 400 of the front and rear chassis (480, 490, respectively) of the ICD with the principal mechanics exposed. Evident is syringe plunger with a rack 453 configuration that is coupled to a worm gear 418 (the axle of which is shown here—the spiral teeth being on the opposite side). Slot(s) 455 are formed in the front chassis 480 that fit the syringe finger rests, restricting longitudinal movement of the syringe (not shown) when placed therein, and to cause the syringe body to move in unison with the ICD body. Worm 315 is coupled to the worm gear 418.

As discussed above, finger actuators 120 are secured to two timing belts 260 and “moves” the timing belts 260 which spin the worm 315 via timing pulleys 355 (covered from view by the timing belts 260) and spin about opposite pulley 355. Opposite pulley 355 is attached to rear chassis 490. While a pair of timing belts 260 are shown, a single timing belt or rigid racks, screw drive, etc. may be used in alternate embodiments. Pawl 375 is fixed to the timing belts 260 by a mounting bracket 416 so that as the timing belt 260 is rotated (spinning counter-clockwise in this FIG.) the pawl 375 moves forward and when engaged with the position reference member 140, holds the position reference member 140 against the tissue as the ICD body is retracted.

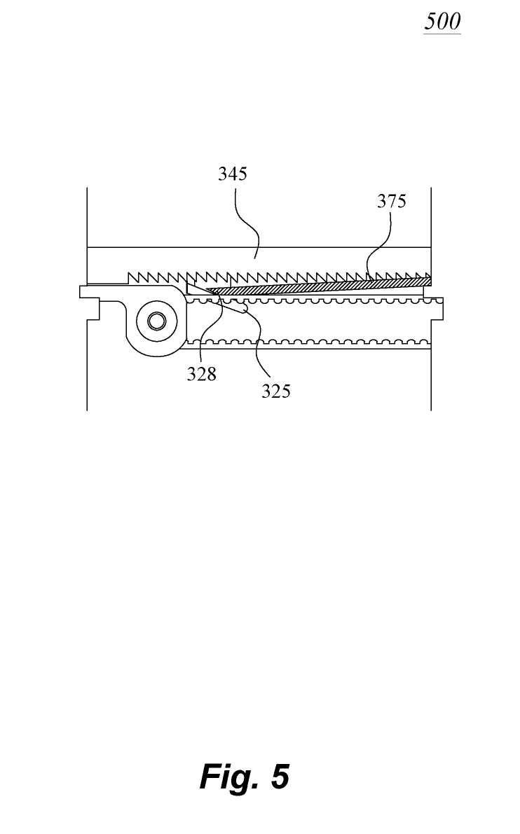

FIG. 5 is a close-up side view of the pawl 375 to ramp 325 arrangement. This FIG. has the finger acutators and chassis components removed for easier viewing. Viewable here, the pawl 375 will engage with angled teeth 345 on the position reference member 140 at its trough or angled terminal end 328. The trough/angled terminal end 328 also allows the position reference member 140 to slide (e.g., ratchet) over the pawl's angled terminal end 328 when the position reference member 140 is extended forward. In operation, the trough/angled terminal end 328 is directed into the teeth 345 via the ramp 325 that the trough/angled terminal end 328 strikes when the pawl 375 is moved forward by timing belt 260. The ramp 325 is fixed to the front chassis 480 (see FIG. 4.) and functions to “elevate” the trough/angled terminal end 328 into angled teeth 345. The ramp 375, position reference member 140 or pawl 375 can be configured to operate as a ratchet mechanism (e.g., the ramp 325, or the pawl 375, can be tilted to allow the pawl 375 and/or position reference member 140 to slide by), if so desired.

It should be understood that the interaction between the transmission system (e.g., timing belt 260) and the position reference member 140 via the pawl 375, alternative engaging systems may be utilized without departing from the spirit and scope of this disclosure. For example, in an alternative embodiment, the pawl 375 or some extension thereof can be used to indirectly force the position reference member 140 forward, via rearward motion rather than directly through a forward motion. That is, the pawl 375 (or some variant of it) could be connected to the finger actuator 120 or the lower portion of the timing belt (rather than upper timing belt), and be moved in a rearward direction in concert with the motion of the finger actuator 120. And the pawl 375 (or variant) could be in the form of a rack that travels rearward with the finger actuator, the rack section rotating a rearward gear (not shown) that is in contact with the teeth 345 of the position reference member 140. Thus, the rearward gear would drive the position reference member 140 forward via force from the rearward moving pawl 375 (or variant).

Additionally, the teeth 345 of the position reference member 140 may be replaced with a mechanism that engages the position reference member 140 in response to the transmission system (e.g., timing belt 260). For example, a friction-based mechanism that adheres to a section of position reference member 140 could be used. For this scenario, the position reference member could be “clutched” to the timing belt 260. Thus, a “clutching” system that grips the position reference member 140 when engaged and releases the position reference member 140 when disengaged could be used in lieu of the transmission-to-pawl-to-position reference member system described. Similarly, the syringe plunger 453 may be “non-teethed” and driven, for example, by a friction wheel(s) or other mechanism from the worm gear assembly, if so desired.

As is apparent, the exemplary transmission-to-position reference member system and auxiliary elements can be achieved by direct or indirect approaches. Therefore, it is understood that while the exemplary configuration is shown as having specific components operating in specific ways, assorted variations, modifications, changes may be to the exemplary drive by one of ordinary skill in art without departing from the spirit and scope of this disclosure.

Returning to FIG. 5, the displayed pawl 375 to ramp 325 arrangement provides the necessary flexibility for the different steps for preparing and injecting. That is, when the ICD and cannula (not shown) is maneuvered by the technician—inserted into the tissue—the position reference member 140 (being in a first extended position) is free to retract into the ICD when its end is pressed against the tissue, since the pawl 375 is not in contact with the position reference member 140 teeth 345. When the injection step is desired, the upper side of the timing belt 260 is urged forward (by finger actuators pulling the lower side of the timing belt 260 counterclockwise) causing the pawl 375 (being coupled to the upper side of the timing belt 260) to move forward (e.g., counterclockwise). The pawl 375 strikes the ramp 325 to move the terminal end 328 into teeth 345, engaging the position reference member 140. After injection, the finger actuators are returned to their initial position via a clock spring or other mechanism, which releases the terminal end 328 of the pawl 375 from teeth 345 of the position reference member 140 (for example, the pawl 375 will drop down into its resting position); freeing the position reference member 140 to also return to its initial position.

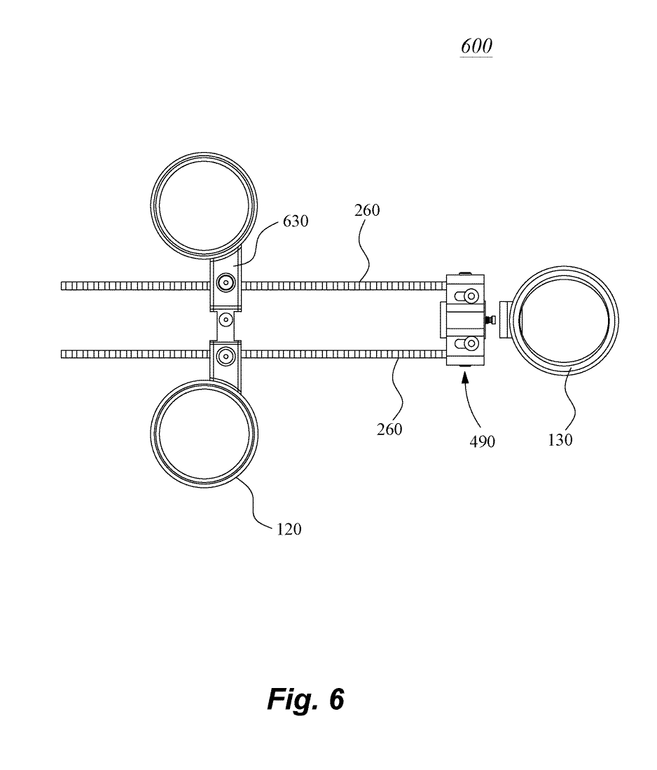

FIG. 6 is an exposed partial view 600 of the timing belts 260 with finger actuators 120 and thumb rest 130. The pair of timing belts 260 rotate around rear pulley (not shown) housed in rear chassis 490. As evident here, thumb rest 130 may be removable and changed with an alternate design, if so desired. Finger actuators 120 as shown here as attached to the timing belts 260 via mounting hardware 630.

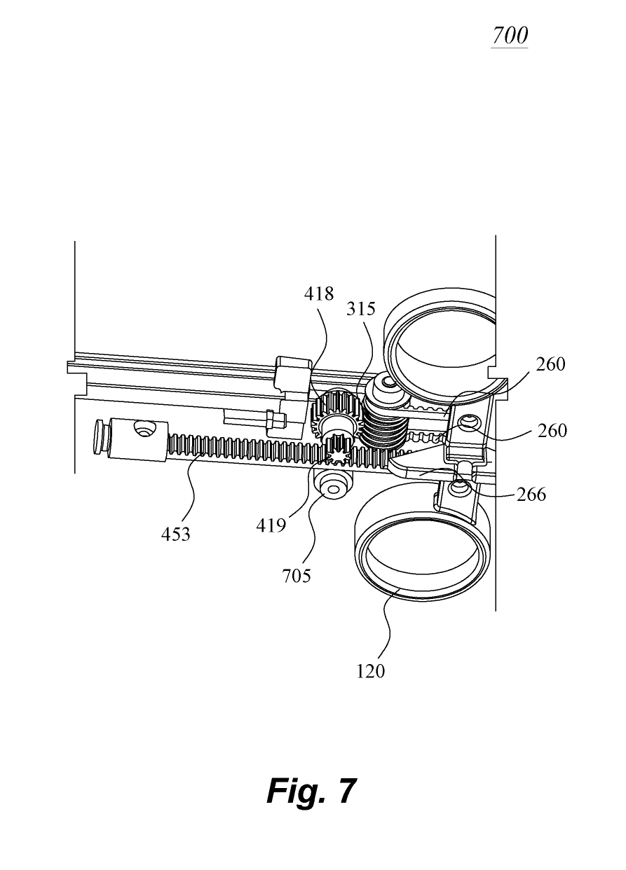

FIG. 7 is a reversal of FIG. 4, with a close-up view 700 of the worm-to-timing belt-to-worm gear configuration. The general mechanics of a worm gear/main gear system are well detailed in U.S. Pat. No. 8,540,681, titled “INJECTION CONTROL DEVICE WITH GEARING MECHANISM” by the instant inventor, the subject matter of which is incorporated by reference herein. The system disclosed herein is a significant modification of the system of U.S. Pat. No. 8,540,681, using a timing belt for driving the worm and a pawl for pushing against the position reference member, thereby allowing for single-hand operation. For the sake of completeness, a brief description of the representative elements is provided.

Finger actuators 120 are fixed to each of timing belts 260, which circle around timing pulleys of worm 315 (it is noted, portions of the two timing belts are obscured from view). Finger actuators 120 are fitted with an optional central guide 266 that is configured to fit within an ICD guide slot so as to stabilize the motion of the finger actuators 120 as it travels through the ICD. As worm 315 turns, it rotates worm gear 418 which is fitted with a drive gear 419 that is in contact with syringe plunger rack 453. A pinch roller 705 is disposed at the back side of the syringe plunger rack 453 to guide its motion.

It should be appreciated that the above examples are illustrative of a possible embodiment for achieving a single hand ICD unit. Various modifications to the above design can be made to achieve different capabilities, according to design preference. For example, instead of using a “gear-driven” syringe plunger, an automatic (e.g., electronic, motored, or springed, etc.) plunger activator may be use. As mentioned above, a single or plurality of timing belts may be used, or a rigid rack or gearing system that provides the equivalent function.

In the various embodiments described, and as further elaborated in U.S. Pat. No. 8,540,681, a clutching arrangement can be utilized to control the directional operation of the worm 315. For example, a first clutch can be fixed to the body of the ICD to only allow unidirectional rotation of the drive gear 419 and a second clutch can be used in the worm 315 to only allow rotation when the timing belt 260 is rotated in an “injection direction.” These clutches prevent the syringe plunger rack 453 from being moved/pushed when the finger actuators 120 are being reset to their initial position.

Various interchangeable tips can be attached to the position reference member 140 to accommodate different cannula types and injection depths. Further, position reference stops can be incorporated into the ICD to accommodate different cannula types, injection depths, and cannula working lengths, and so forth.

By use of the ICD, several advantages can be obtained in one or more of the various embodiments:
The injection of the filler material is substantially proportional to the length of the injection tract and uniform along the course of the injection tract;
An “automatic” controlled injection system can be used for fat grafting or injection of other filler materials;
Intracutaneous, subcutaneous and intramuscular injections of filler materials can be precisely controlled;
A fixed amount of fat or other filler material can be injected per unit distance traveled by the tip of the cannula;
The injection ratio (amount of material injected over a given distance of cannula withdrawal) can be varied by simply using varying bore diameter syringes;
The use of syringes (disposable);
The use of syringes incorporating a rack in the plunger;
For the meat or food industry, injecting juices, fluids, flavors and so forth can be distributed within the meat, rather than pooled into a specific area.
Filler materials can be organic, inorganic, living or non-living materials, chemical solutions and so forth.

It should be appreciated that based on an understanding of various embodiments of the injection control device disclosed herein, several modifications may be contemplated without departing from the spirit and scope of this disclosure. As some cannulas may be of different diameters and openings, a volume approach may be achieved by adjusting the gearing, for example.

In various applications, it is envisioned that using a cylindrical cannula will result in cylindrical tracks of material left in the channel created by the cannula's intrusion. Using computer/automated devices, an increased degree of control can be obtained in the amount and “shape” of the deposited material or extracted material as well as variation of the injection/extraction profile. For example, conical, elongated spheres, or series of spheres could be produced. A similar result can also be obtained by using a camming system in the transmission system to periodically delay/increase the rate of injection/extraction. Following this, a robotic system which precisely controls the position and rate of motion of the cannula and/or rate of injection/extraction could be implemented in the ICD. Moreover, while the “applications” are in the context of a cannula “inside” a subject, the ICD can be easily adapted to regulate the rate of extrusion of a fluid for a topical application.

It will be understood that many additional changes in the details, materials, steps and arrangement of parts, which have been herein described and illustrated to explain the nature of the disclosure, may be made by those skilled in the art within the principle and scope of the disclosure as expressed in the appended claims.

What is claimed is:

1. An injection control device, comprising:
a body have a forward and rearward end;
a syringe supporting section coupled to the forward end of the body;
a gear assembly coupled to the body, and configured to actuate a prospective syringe plunger when the gear assembly is driven;
a transmission system coupled to the body and to the gear assembly, providing a driving force when actuated;
at least one transmission system actuator, coupled to the transmission system;
an extendable, automatically self-positioning, position reference member (PRM), a forward portion having a reference end for contact with a subject surface and extending from the forward end of the body; and
a transmission-to-PRM engagement system coupled to the transmission system and coupled to the PRM,
wherein when the actuator is motioned in a designated direction, the transmission system drives the gear assembly to actuate the prospective syringe plunger into a prospective syringe barrel held in the syringe supporting section and drives the transmission-to-PRM engagement system to actuate the position reference member against the subject surface, causing the body and prospective syringe to move away from the subject surface, these combination of motions resulting in an injectate to be deposited in proportion to a function of the position reference member travel or prospective syringe cannula travel.

2. The injection control device of claim 1, wherein the transmission is comprised of a pulley coupled to the rearward end of the body, and at least one timing belt coupled to the gear assembly and to the pulley.

3. The injection control device of claim 1, wherein the transmission-to-PRM engagement system is comprised of:
a pawl with an angled end directed to the forward end of the body, coupled to the transmission system, configured to move forward when the transmission system is activated via motion of the actuator; and
a ramp coupled to the body, causing the pawl, when moved forward, to engage the PRM.

4. The injection control device of claim 3, wherein a PRM has angled teeth.

5. The injection control device of claim 1, further comprising a syringe with a cannula held by the injection control device.

6. The injection control device of claim 1, further comprising:
at least one spring coupled to the transmission system and the PRM; and
a gear assembly release,
wherein as the actuator is released, the actuator and the PRM automatically return to a starting position and the prospective syringe plunger remains stationary.

7. The injection control device of claim 5, further comprising a toothed syringe plunger in contact with the gear assembly.

8. The injection control device of claim 1, wherein the gear assembly comprises a worm and worm gear.

9. The injection control device of claim 8, wherein the worm is a fixed direction worm.

10. The injection control device of claim 9, wherein the fixed direction is via a clutch.

11. The injection control device of claim 2, further comprising a spring in the pulley, providing a restorative force to bring the actuator into an initial resting position.

12. The injection control device of claim 1, wherein the actuator is adapted to fit a finger and configured to travel though a guide slot in the body.

13. The injection control device of claim 2, wherein the timing belt is a plurality of timing belts.

14. The injection control device of claim 1, further comprising a thumb hold disposed at the rearward end of the body.

15. The injection control device of claim 1, wherein the reference end of the PRM is positioned over the prospective cannula.

16. The injection control device of claim 1, wherein actuator enables operation with a single hand.

17. The injection control device of claim 3, wherein at least one of the pawl and ramp functions as a ratchet.

18. The injection control device of claim 1, wherein the injectate is at least one of a fat, filler, flavor, oil, liquid, living cells, and chemical.

19. The injection control device of claim 1, further comprising:
a guide slot in the PRM; and
a stop disposed in the body and in the guide slot, controlling a range of motion of the PRM.

20. The injection control device of claim 19, wherein the stop is removable.
